(19) United States
(12) Patent Application Publication
(10) Pub. No.: US 2006/0116720 A1
(43) Pub. Date: Jun. 1, 2006

(54) Method and apparatus for improving renal function

(76) Inventor: Penny Knoblich, Mankato, MN (US)

Correspondence Address: BECK AND TYSVER P.L.L.C., 2900 THOMAS AVENUE SOUTH, SUITE 100, MINNEAPOLIS, MN, 55416, US

(21) Appl. No.: 11/001,423

(22) Filed: Dec. 1, 2004

(51) Int. Cl. A61N 1/00

(52) U.S. Cl. 607/2

(57) ABSTRACT

An electrical stimulator for providing electrical energy to nerves related to renal function for the purpose of improving and controlling renal function.

### FIELD OF THE INVENTION

The present invention relates generally to electrical stimulation of body tissue for a therapeutic effect on the kidneys, and more particularly to a technique for increasing sodium excretion by the kidneys.

### BACKGROUND OF THE INVENTION

The kidneys are essential organs located at the back of the abdomen on each side of the spinal column at about the level of the lower ribs. The kidneys receive about 20% of the cardiac output. They function to remove waste products from the blood and regulate blood electrolytes, acid-base balance, total body water, and blood volume.

The basic functional unit of the kidney is the nephron. Each nephron includes a glomerulus, a capillary through which blood flows and from which fluid is filtered. Filtered fluid enters the tubules, which process the fluid. At the end of the tubules, the filtered fluid ultimately becomes urine. The standard measure of renal function is the glomerular filtration rate, or the total rate that fluid is filtered from all the glomeruli combined. The normally functioning kidney controls the blood electrolytes, acid-base balance, total body water, and blood volume by adjusting the reabsorption (back into the body) or secretion (from the body into the filtered fluid) of electrolytes, acids and bases, and water. If excess water is present, it is excreted in the urine. If excessive solutes are present, they are excreted preferentially. In spite of large intakes of either water or salt, the normal kidney can accommodate and precisely regulate the volume and composition of the blood.

Although the primary measure of kidney function relates to these excretory functions, it is important to note that the kidneys also function to produce hormones. The kidneys are in part responsible for the conversion of Vitamin D to its active metabolite, a hormone that functions to increase the absorption of calcium from the intestines. The kidneys also synthesize erythropoietin, a stimulating hormone for red blood cell production, and renin, a hormone involved in the regulation of sodium reabsorption and the maintenance of blood pressure.

Proper elimination of sodium from the body is one of the critical functions of the kidney. Failure of the kidneys to adequately eliminate sodium increases total body sodium and water, and blood volume. The increase in blood volume raises pressure in the vascular system, producing hypertension, or high blood pressure.

Progressive renal failure, occurring as a result of a variety of disorders, can give rise to a number of symptoms which decrease both the length and quality of life. Vascular damage to the glomeruli, infiltration of the renal tissues with inflammatory cells, and damage and scarring of the tubules, all contribute to the degeneration of renal function. Pathological processes primarily affecting the vasculature, such as diabetes mellitus, hypertension, overuse of over-the-counter anti-inflammatory medications, and side-effects of some pharmaceutical agents, preferentially damage the glomeruli. Chronic inflammation, repeated infection, or certain poisons may damage the tubular system. Renal failure is typically characterized by a progressive inability to maintain normal electrolyte composition, blood pH, and body water volume. Sodium chloride, or salt, becomes progressively more difficult to eliminate from the body. Concomitant interactions increase blood volume and pressure, cause acidosis, and produce edema in body tissues.

In many instances, the treatment of kidney failure attempts to address the secondary symptoms, rather than directly impact the function of the kidneys themselves. Diuretics to reduce blood volume, pain medication, and other pharmaceutical agents directed at alleviating the secondary effects are commonly used. End stage kidney disease is typically treated by hemodialysis, in which the blood is “cleaned” by exchange with a dialysis fluid across a selectively permeable membrane. Given the wide range of important functions, it is desirable to develop methods and devices to alter kidney function both prior to and following significant renal disease or damage in cases of renal degenerative disorders, or systemic pathology likely to result in renal damage or degeneration.

### SUMMARY OF THE INVENTION

In contrast to pharmacological treatments of renal dysfunction and hemodialysis as a treatment for end stage renal disease, the present invention relates to the use of electrical stimulation of specific nerve pathways to alter and improve renal function. The method and apparatus has been implemented in an animal model using an extracorporeal generator. Human treatment modalities and implantable electrical stimulator embodiments are anticipated.

### DETAILED DESCRIPTION

The essential organs of the body are interconnected in an elaborate control system that involves afferent (into the spinal cord) and efferent (spinal cord to organ) nerves, coupling the organ with the spinal cord. Organ function depends not only upon electrochemical signals through these neurological pathways, but also on cytokines and other signal molecules produced locally within the kidney, or circulating in the blood. Although electrical stimulation of body tissues is well known, the complexities of organ function make it difficult to fully predict or understand the impact of either pharmaceutical or electrical therapy on a given organ system.

Overview of Experimental Design

Applicants have applied electrical stimulation in a rat model to the dura mater (a protective membrane surrounding the spinal cord) on the dorsal surface of the spinal cord, in the area of the spine in which renal sensory afferent nerves enter and interact with other nerves of the spinal cord.

A variety of stimulation regimes were applied to the rat model and the effect of stimulation noted as a function of time. Certain stimulation patterns and specific stimulation regimes result in substantial increases in the excretion of sodium when compared to baseline amounts, or sham-treated rats. The interaction of the stimulation and post-stimulation has been explored.

Experimental Data

The theory of interaction is an effort to explain the experimental results but it may be wrong or incomplete. Applicants predicate patentability in part on the surprising effectiveness of certain stimulation patterns.

The sympathetic nervous system is a part of the autonomic nervous system, which controls involuntary functions. Among other effects, the sympathetic nervous system has been shown to reduce total renal blood flow, increase renin release (resulting ultimately in an increase in the reabsorption of sodium), and change the distribution of blood flow between the outer renal cortex and inner medulla (altering sodium reabsorption). The sympathetic nerve supply to the kidney arises from the ipsilateral paravertebral sympathetic nerve ganglia in the area between the thoracic segment (T11) and the lumbar segment (L3). The afferent (sensory) myelinated renal nerves carry information from intrarenal receptors, and enter the spinal cord via the dorsal root at spinal level T11-T12 for the left kidney, and T9-T10 for the right kidney. Incoming sensory information travels in the dorsal column system of the spinal cord to both visceral afferent and dorsal column nuclei. Numerous interactions between these sensory nerves, the sympathetic nervous system, and efferent nerves to the kidney are described. This representation of the rat anatomy is required to understand the location of the stimulation electrodes.

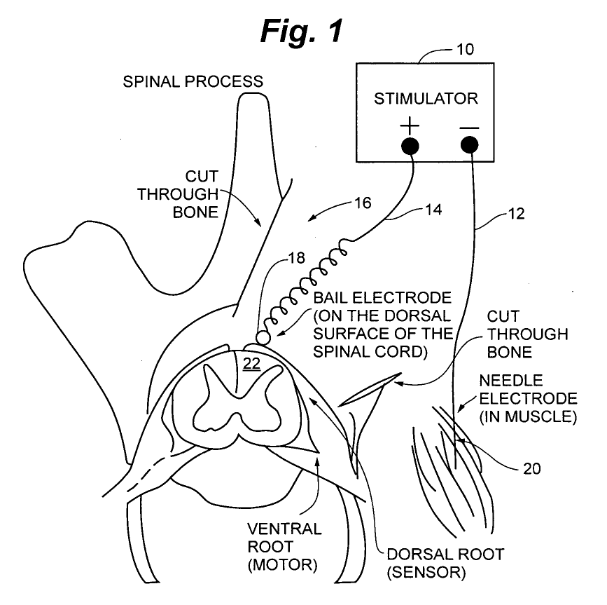

FIG. 1 depicts the design of the experiment, where a portion of the subject rat's spinal process is removed creating a surgical passage 16. The stimulator 10 is coupled though two electrode leads 12 and 14 to electrodes 18 and 20. The ball electrode (cathode) 18 is placed on the dura mater of the dorsal surface of the spinal cord 22 and the reference needle electrode (anode) 20 is placed in nearby muscle tissue.

Electrical stimulation was supplied to the electrodes through the programmable stimulator 10. The stimulus was applied in a square wave pattern with a frequency of 50 Hz, and a duration of 0.2 milliseconds. In general, the stimulus strength (voltage) was determined by initially finding the motor threshold, which is the minimum voltage associated with activation and contraction of muscle fibers in the area. The motor threshold was determined by slowly increasing the voltage until contraction of the area musculature (paravertebral muscles) was evident. A variety of stimulation regimes, using differing percentages of the motor threshold were explored. Repeated experiments confirmed and demonstrated that stimulation both near the motor threshold and far below it were less effective at producing an increase in sodium excretion. However, in the experimental model, electrical stimulation corresponding to approximately 67 percent of the motor threshold provided a dramatic increase in sodium excretion (micromoles per kilogram per minute) that extended well beyond the cessation of the stimulus.

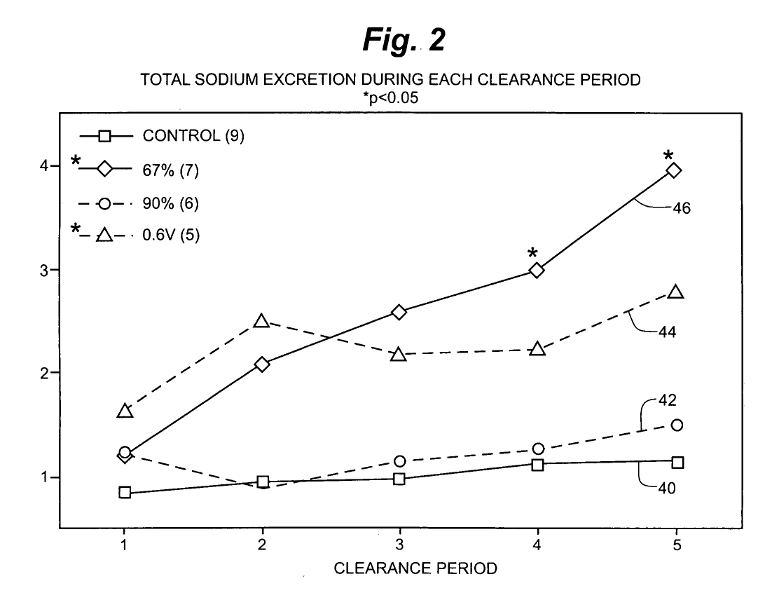

These departures from baseline are set forth in FIG. 2, showing the time course of sodium excretion. In FIG. 2, the control is shown as trace 40, in which no electrical stimulation is applied to the spinal cord of the subject rat. Trace 42 represents a stimulation voltage very near the motor threshold. In this trace, the applied stimulus voltage was 0.90 of the motor threshold. Trace 44 represents the sodium excretion in response to a stimulation voltage of 0.6 volts, a level approximately 0.34 of the mean motor threshold of 1.8 volts. Trace 46 represents the sodium excretory response to a stimulation voltage of 0.67 of the motor threshold. It is important to note that the electrical stimulation was applied only during the second 15-minute collection period, but the effect on sodium excretion lasted a substantially longer time.

Discussion of Results

The theory of interaction is an effort to explain the experimental results but it may be wrong or incomplete. Applicants predicate patentability in part on the surprising effectiveness of certain stimulation patterns.

The mechanism of action is theorized at the present time to involve an increase or redistribution of blood flow in the kidney, permitting the nephrons to decrease the reabsorption of sodium, increasing its excretion rate. Further effects of the electrical stimulus may include an alteration in the level of sympathetic nervous system stimulation, either at the level of the kidney alone, or centrally. Furthermore, retrograde activation of sensory nerves may result in release or production of additional chemicals within the kidney cells.

What is claimed:

1. A device for electrical stimulation to improve kidney function, comprising of:
a) one or more electrodes located at the level of the spine proximate to the afferent and efferent nerves associated with renal function;
b) a pulse generator coupled to the electrodes for generating a current pulse at a voltage of greater than 0.3 but less than 0.90 percent of the motor threshold, and stimulus duration sufficient to stimulate a change in renal function.

2. A method of improving renal function comprising the steps of:
a) placing an electrode (cathode) proximate to the dorsal portion of a spinal cord at the level of the entry of the renal nerves;
b) placing a reference electrode (anode) proximal to the spinal cord to direct current though the nerves of the kidney;
c) applying a stimulation voltage between 0.30 and 0.90 of the motor threshold of the paravertebral muscles at the electrode locations.
